(19) United States
(12) Patent Application Publication
(10) Pub. No.: US 2018/0056055 A1
(43) Pub. Date: Mar. 1, 2018

(54) TATTOO MACHINE WITH OFFSET GRIP

(71) Applicant: Shea Rutherford, Fort Wayne, IN (US)

(72) Inventor: Shea Rutherford, Fort Wayne, IN (US)

(21) Appl. No.: 15/686,978

(22) Filed: Aug. 25, 2017

Related U.S. Application Data
(60) Provisional application No. 62/380,549, filed on Aug. 29, 2016.

(51) Int. Cl. A61M 37/00

(52) U.S. Cl. CPC A61M 37/0076; A61M 2205/586

(57) ABSTRACT

A tattooing system includes a needle defining a needle axis; a needle driver connected to the needle and configured to reciprocally drive the needle along the needle axis; and a grip linked to the needle and defining a grip axis which is non-coaxial with the needle axis.

### CROSS REFERENCE TO RELATED APPLICATIONS

This is a non-provisional application based upon U.S. provisional patent application Ser. No. 62/380,549, entitled “TATTOO MACHINE WITH OFFSET GRIP”, filed Aug. 29, 2016, which is incorporated herein by reference.

### BACKGROUND OF THE INVENTION

### 1. Field of the Invention

The present invention relates to tattoo machines, and, more particularly, to accessories for tattoo machines.

### 2. Description of the Related Art

There are many difficulties that tattoo artists face while creating a tattoo. Some of the difficulties include the artist having a limited line of sight of the tattoo area, a restricted range of motion, and a difficult time holding the tattoo machine steady throughout the tattooing process. These difficulties are partially due to the standard setup of a tattooing system.

The standard setup of a tattooing system is a linear setup including a tattoo machine with a grip connected to a needle driver and a needle driven by the needle driver. The needle driver is typically on one side of the grip and the needle is on the other side of the grip. The needle driver is a piece of equipment that uses a motor, electromagnetic coils, or pressurized air to drive the tattoo needle in a reciprocating motion and deposit ink beneath the surface of the skin. The tattoo grip allows a tattoo artist to comfortably hold the needle driver and needle while creating the tattoo. The tattoo needle is a sharp, pointed rod which can be dipped in a well of a marking substance or can connect to a reservoir of a marking substance to load the needle with the marking substance, which may be ink, with reciprocating movement of the needle piercing the skin and driving the ink into the appropriate layer of the skin. The applied ink is immobilized in the skin, forming a mostly permanent marking in the skin that is visible through the uppermost layers of the skin. The ink may be a variety of different colors which are visible to the naked eye under normal lighting conditions or may only be visible when exposed, for example, to black light or other special lighting conditions. The marking may take the form of various artwork designs or, in some instances, may be formed to replicate or enhance the look of various body structures such as hair, lips, eyebrows, or nipples.

The linear setup of the tattoo system poses difficulties for a user because the needle driver can obstruct vision of the area being tattooed by the user, forcing the user to change the angle of the tattoo machine relative to the skin to obtain a better line of sight of both the tip of the needle and the insertion point in the skin. A typical setup also limits the user's range of motion. When creating a tattoo, the machine will hit the surface of skin and prevent the user from being able to perform needle strokes from certain angles. This can make tattooing more difficult for the user.

Further, range of motion is a concern when tattooing. Some grips are larger in diameter and increase the range of motion. Having a tattoo machine without a large grip installed limits range of motion while tattooing. However, increasing the diameter of the grip can be uncomfortable for many users to hold, especially during a long tattooing session, and also increases the weight that the user must maneuver to create the tattoo, which can make it more difficult to create the desired design.

While creating a line of a tattoo, there are many technique variations. For example, for when creating a broad line the user may insert the needle into the skin at an angle, with angles of thirty to sixty degrees being a median range for creating a line. Forming a smaller angle between the needle and the skin creates a thicker line, while a larger angle, up to 90 degrees, creates a thinner line. Therefore, when a line is being created, the user holds the needle at the desired angle as the line is being created at a controlled speed. This process of tattooing a line takes a lot of practice and skill to achieve, and can fatigue the user.

What is needed in the art is a way to increase user comfort, vision, and accuracy while creating a skin marking.

### SUMMARY OF THE INVENTION

The present invention provides a grip for a tattooing system which is offset from a needle axis defined by a needle of the tattooing system.

The invention in one form is directed to a tattooing system including a needle defining a needle axis; a needle driver connected to the needle and configured to reciprocally drive the needle along the needle axis; and a grip linked to the needle and defining a grip axis which is non-coaxial with the needle axis.

The invention in another form is directed to a method of creating a marking in a skin layer of an animal using a tattooing system including a needle defining a needle axis and a needle driver connected to the needle. The method includes: loading the needle with a marking substance; recriprocatingly driving the needle with the needle driver; holding a grip linked to the needle and defining a grip axis which is non-coaxial with the needle axis; and moving the grip such that the needle loaded with the marking substance goes into the skin of the animal to deposit the marking substance in the skin layer.

One possible advantage of the previously described tattooing system is that offsetting the grip axis from the needle axis can make it easier for a user to visualize the needle while creating a mark in the skin of an animal.

Another possible advantage of the previously described tattooing system is the grip can reduce the fatigue experienced by a user while creating a marking in the skin of an animal.

Yet another possible advantage of the previously described tattooing system is the offset angle between the needle and the grip can be adjustable to give a user a wide variety of holding positions relative to the needle.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a perspective view of a prior art tattooing machine;

FIG. 2 is a perspective view of the tattooing machine shown in FIG. 1 being held by a user;

FIG. 3 is a perspective view of an embodiment of a grip formed according to the present invention;

FIG. 4 is a perspective view of the grip shown in FIG. 3 connected to the tattooing machine shown in FIGS. 1-2;

FIG. 5 is a perspective view of the tattooing machine and grip shown in FIG. 4 being held by a user;

FIG. 6 is a perspective view of the tattooing machine and grip shown in FIGS. 4-5 being used to form a marking in the skin of an animal; and

FIG. 7 is a perspective view of an alternative embodiment of a grip formed according to the present invention which may connect to the tattooing machine shown in FIGS. 1-2.

Corresponding reference characters indicate corresponding parts throughout the several views. The exemplification set out herein illustrates one embodiment of the invention and such exemplification is not to be construed as limiting the scope of the invention in any manner.

### DETAILED DESCRIPTION OF THE INVENTION

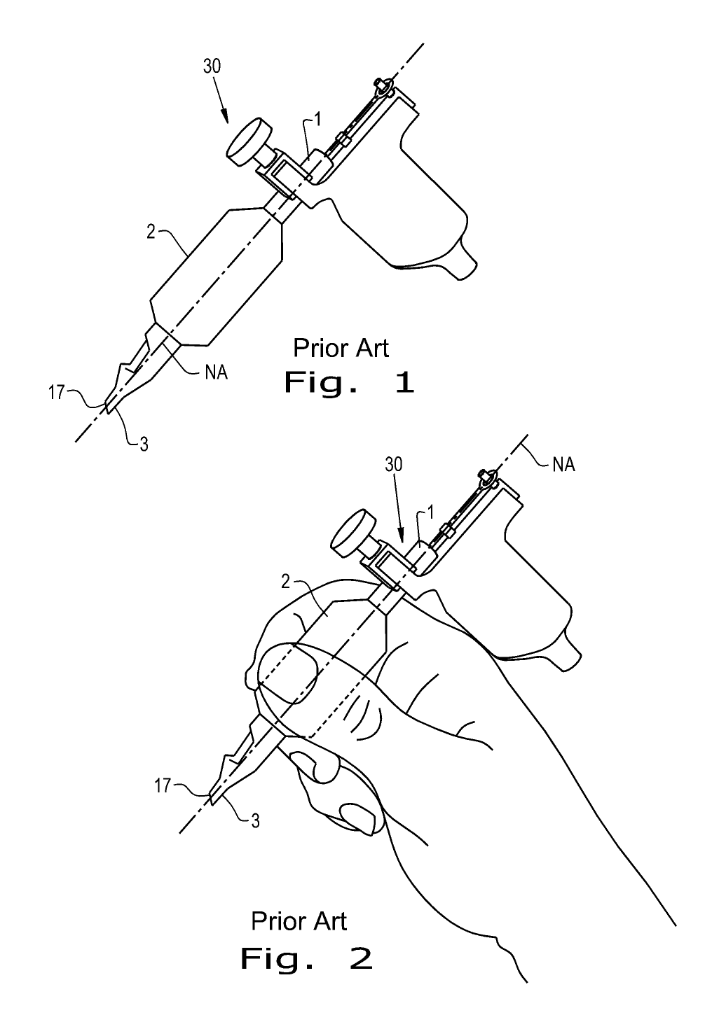

Referring now to the drawings, and more particularly to FIGS. 1-2 there is shown a tattooing machine 30 of the prior art which generally includes a needle driver 1, a needle 3 connected to the needle driver 1 which defines a needle axis NA, and a gripping portion 2 between and connected to the needle driver 1 and needle 3. The needle driver 1 is configured to reciprocally drive the needle 3 along the needle axis NA in order to deliver ink from the needle 3 under a skin surface to create a marking. The needle 3 can have a well 17 formed therein that will fill with a marking substance, such as tattooing ink, when the needle 3 is dipped into a reservoir of ink (not shown in FIGS. 1-2) and which will then be delivered under the skin surface to a desired skin layer in an animal. The needle 3 can have any type of construction suitable to deliver ink or other marking substance(s) and create a marking, such as a tattoo, in the skin of a human or non-human animal, with many such constructions being known. It should be appreciated that while ink is described further herein as being the marking substance delivered under the skin surface of an animal, other substances which may or may not be marking substances can also be delivered into the skin according to the present invention. The needle driver 1 can connect to the needle 3 in any suitable way to create reciprocating motion of the needle 3 along the needle axis NA. Further, the needle driver 1 can utilize any suitable way to create reciprocal motion of the needle 3 along the needle axis NA to deposit ink under a skin surface such as a linear motor, electromagnetic coils, or pressurized air.

As can be seen, the gripping portion 2 is a mostly cylindrical shape defined about the needle axis NA, i.e., the needle axis NA also defines a second grip axis of the gripping portion 2 which is coaxial with the needle axis NA. In this sense, the user can hold the gripping portion 2 like a pen or pencil, with the tip being the needle 3 that delivers ink under a skin surface. While creating the tattoo for a client, a user will typically hold the gripping portion 2 similarly to the way shown in FIG. 2 and then draw the tattoo in a similar fashion to how the tattoo would be drawn with a pen or pencil. However, due to the need for the needle driver 1 to drive the needle 3, the needle driver 1 has a relatively large construction that can obstruct the view of the user during the tattooing process. Further, the radius of the needle 3 is typically very small compared to the rest of the components of the system in order to precisely deliver ink under a skin surface. Such a small radius of the needle 3 makes it difficult for a user to visualize the needle 3 during the tattooing process if other components of the tattooing machine 30 are in the user's field of vision.

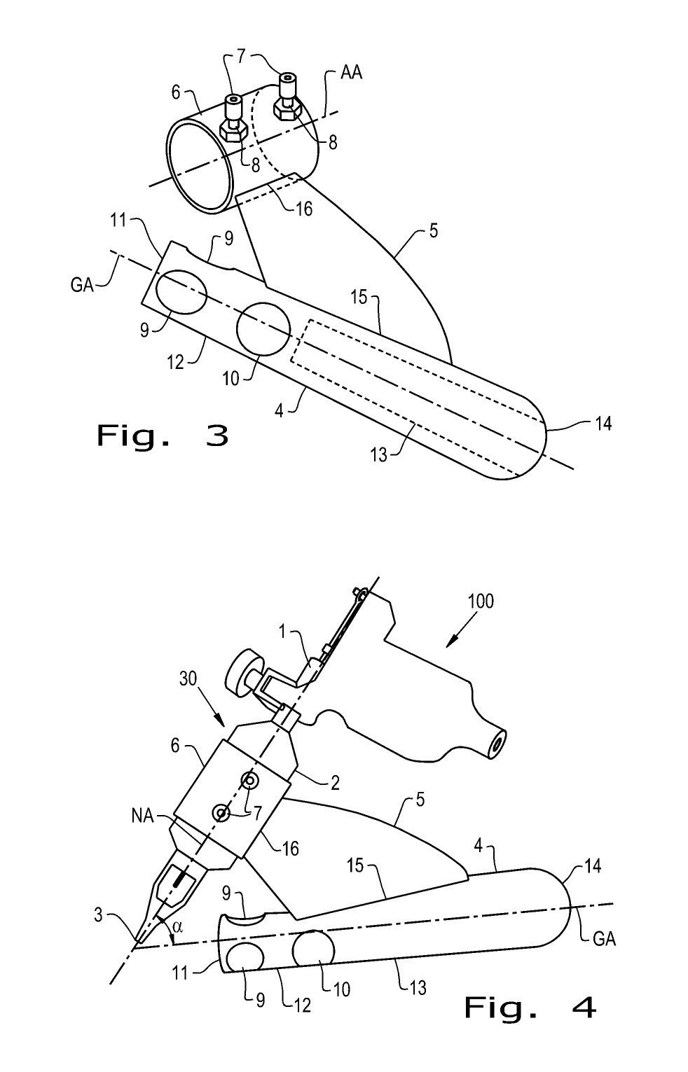

To overcome some of the aforementioned problems, and referring now to FIGS. 3-4, a grip 4 defining a grip axis GA is linked to the needle 3 to form a tattooing system 100 (shown in FIG. 4). As used herein, the grip 4 is “linked” to the needle 3 in the sense that a user holding the grip 4 can manipulate movement of the needle 3 by moving the grip 4. The grip 4 can, for example, connect to the gripping portion 2 of the tattooing system 100 so the user can manipulate the needle 3 using the grip 4. The grip axis GA of the grip 4 is non-coaxial with the needle axis NA of the needle 3 so the grip axis GA, and therefore the grip 4, is offset from the needle axis NA defined by the needle 3. As shown, the grip axis GA of the grip 4 is the longitudinal axis of the grip 4, with the grip 4 having a substantially cylindrical shape defined about the grip axis GA, but the grip 4 can have other shapes so long as the grip 4 can be held and manipulated by a human hand. By offsetting the grip 4 from the needle 3, the user is better able to visualize the needle 3. The grip 4 can include first indentations 9 spaced evenly from one another around the grip 4 about the grip axis GA and second indentations 10 spaced evenly from one another around the grip 4 about the grip axis GA. Both sets of indentations 9 and 10 are shaped and provided to increase user comfort during the tattooing procedure. The first indentations 9 can be formed closer to a first end 11 of the grip 4 which will be closer to the needle 3, when the grip 4 is linked to the needle 3, to provide a gripping position for a user that emphasizes stability and control of the needle 3, whereas the second indentations 10 can be formed farther from the first end 11 to emphasize a wider range of motion for the user and a free-flowing stroke technique. It should be appreciated that other indentations can be formed in the grip 4 and the placement of the shown indentations 9 and 10 can be adjusted as desired.

As shown, the tattooing system 100 can include a bridge portion 5 connecting the grip 4 to an adapter 6. The bridge portion 5 may include, for example, a first bridge end 15 which connects to the grip 4 and a second bridge end 16 which connects to the adapter 6, connecting the grip 4 to the adapter 6 and therefore linking the grip 4 to the needle 3. The adapter 6 can have a complementary shape to the gripping portion 2 in order to fit over the gripping portion 2 of the tattooing machine 30 shown in FIGS. 1-2, as shown in FIG. 4, to allow the grip 4 to retrofit to the tattooing machine 30; as shown, the adapter 6 has a cylindrical shape which complements the cylindrical shape of the gripping portion 2, but it should be appreciated that the adapter 6 can have different complementary shapes to non-cylindrical gripping portions. In some exemplary embodiments, the bridge portion 5 and adapter 6 may be integrally formed with the grip 4; in other exemplary embodiments, the bridge portion 5 and adapter 6 may be separable from the grip 4.

To secure the adapter 6 to the gripping portion 2, the adapter 6 can include one or more fasteners, shown as threaded screws 7, configured to tighten the adapter 6 to the gripping portion 2. The threaded screws 7, for example, can advance through threaded holes 8 toward an interior of the adapter 6. The threaded screws 7 will, when advanced sufficiently within the interior of the adapter 6, abut against the gripping portion 2 and provide an interference fit between the adapter 6 and the gripping portion 2 to secure the adapter 6 to the gripping portion 2, tightening the adapter 6 to the gripping portion 2. It should be appreciated that the shown way of attaching and tightening the adapter 6 to the gripping portion 2 is exemplary only and the adapter 6 can be attached to the gripping portion 2 in any suitable way that links the grip 4 to the needle 3.

As shown in FIG. 4, the adapter 6 can be defined about an adapter axis AA (shown in FIG. 3) which will be coaxial with the needle axis NA when attached to the gripping portion 2. In this sense, the orientation of the grip axis GA relative to the adapter axis AA will define the orientation of the grip axis GA relative to the needle axis NA when the adapter 6 is attached to the gripping portion 2 in order to link the grip 4 to the needle 3. The configuration of the bridge portion 5 and grip 4 can thus be adjusted accordingly to produce the desired orientation of the grip axis GA relative to the needle axis NA. As shown in FIG. 4, for example, the grip 4 and bridge portion 5 can be configured such that the grip axis GA and needle axis NA form an acute angle α when the grip 4 is linked to the needle 3. The angle α formed by the grip axis GA and needle axis NA can be adjusted, as desired, to user specifications, with angles between 40 and 70 degrees having been found to be useful values for tattooing. Other angles α formed by the grip axis GA and needle axis NA may also be useful for tattooing or other types of skin marking, such as micropigmentation to replicate the look of missing hair on a scalp. In some exemplary embodiments, the adapter 6 has padding (not shown) or other materials attached to the interior of the adapter 6 which can dampen vibrations created by the needle driver 1 during a procedure and lessen the discomfort experienced by the user. It should be appreciated that while the grip 4 shown in FIG. 4 is linked to the needle 3 by the adapter 6 in order to retrofit the grip 4 to an existing system, the grip 4 can alternatively be formed integrally with a housing of the needle 3. It should be further appreciated that while the angle α formed by the grip axis GA and needle axis NA is shown as being a fixed angle, the bridge portion 5 can be configured to include a pivot, hinge, or other mechanism that allows a user to adjust the angle α formed by the grip axis GA and needle axis NA, as desired. By offsetting the grip axis GA from the needle axis NA, the user's vision of the area being tattooed can be increased since the needle driver 1 will be less likely to block vision of the area. The range of motion of the user can also be increased without needing to increase the diameter of the grip 4, allowing for increased range of motion without the discomfort associated with increasing the diameter of the grip 4 to a large extent.

To further increase the comfort of a user during a tattooing procedure, the grip 4 can have a weight distribution that places the center of gravity of the grip 4 toward the first end 11 of the grip 4, which will be closer to the needle 3 during use. This can be done, for example, by the grip 4 having a solid portion 12 formed adjacent to the first end 11 and the rest of the grip 4 being a hollowed portion 13, with the hollowing formed in the grip 4 being shown by phantom lines in FIG. 3. By forming the grip 4 in this fashion, the center of gravity of the grip 4 can be located close to the first end 11 which is closer to the needle 3 during use. By having the center of gravity of the grip 4 closer to the first end 11 in combination with the offset of the grip 4, the weight of the grip 4 can provide the pressure needed to press the linked needle 3 into the surface of the skin and deliver ink. This eliminates the need for a user to press the needle 3 into the skin surface during the tattooing process, decreasing the strain a user may experience during the process and also making it easier to keep the needle 3 at a consistent penetration depth in the skin surface during the tattooing process. The solid portion 12 can also be angled relative to the hollowed portion 13, allowing the grip 4 to be easily held like a pen or pencil, as shown in FIG. 5. While the tattooing system 100 shown in FIG. 5 is arranged for a right-handed user, the system can be easily adapted for a left-handed user by arranging the system as a mirror image of the system shown in FIG. 5. The hollowed portion 13 can also be tapered, i.e., increase in diameter, from the first end 11 toward a second end 14 opposite the first end 11 to increase comfort and accommodate users with larger hand sizes. Further, the grip 4 can be formed of materials commonly used to make current grips, such as stainless steel, aluminum, titanium, graphite, and various polymers, with it being understood that the material used to form the grip 4 is readily changeable in the spirit of the present invention.

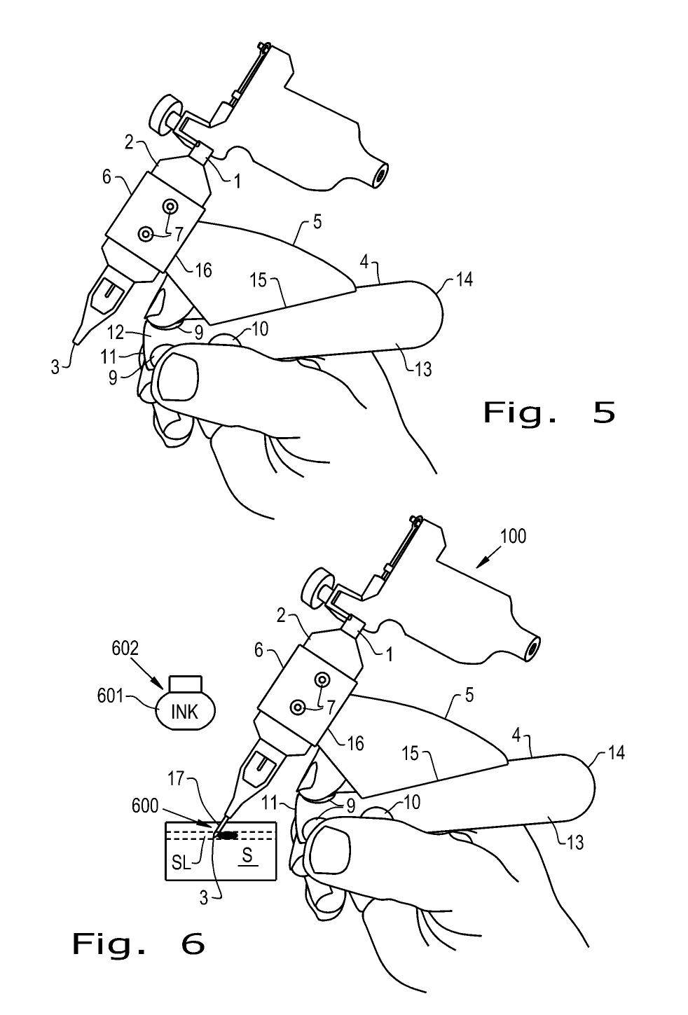

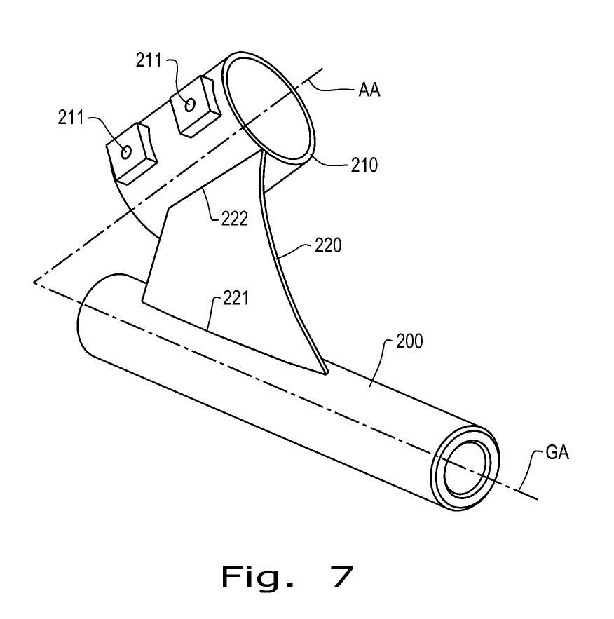

Referring now to FIG. 6, the tattooing system 100 shown in FIGS. 4-5 is shown being used to create a marking 600 in the skin S of an animal. To form the marking 600, the needle 3 can be loaded with a marking substance 601, such as a colored ink. To load the needle 3 with the marking substance 601, the needle 3 can be dipped in a container 602 of the marking substance 601 so the well 17 of the needle 3 is filled with and holds the marking substance 601. The needle driver 1 can be activated to reciprocatingly drive the needle 3 along the needle axis NA; the needle 3 can be reciprocated before, during, and/or after being loaded with the marking substance 601. A user holds the grip 4 linked to the needle 3, as shown, and moves the grip 4 such that the needle 3 loaded with the marking substance 601 pierces and goes into the skin S of the animal to deposit the marking substance 601 in a skin layer SL of the animal. The skin layer SL may be, for example, the dermal layer of the skin S just below the epidermal (outer) layer of the skin S. The user may move the grip 4, and linked needle 3, as much as desired to create the marking 600 in the skin, reloading the needle 3 with marking substance 601 as needed or desired; in some exemplary embodiments, the marking 600 may be completed by the user in one session or the user may take multiple sessions to complete the marking 600. Further, the marking 600 may be formed as an artistic marking or, in some embodiments, may be formed to replicate or enhance body structures of the animal such as missing hair on a scalp, eyebrows, lips, nipples, etc. Therefore, it should be appreciated that the tattooing system 100 and associated method provided according to the present invention may be used in a variety of ways to create different markings in the skin of a human or non-human animal.

Referring now to FIG. 7, an alternative embodiment of a grip 200 formed according to the present invention is shown which may be used in place of the previously shown and described grip 4. As can be seen, the grip 200 defines a completely hollowed cylinder about a grip axis GA which is the longitudinal axis of the grip 200. The cylindrical grip 200 may, in some embodiments, have a uniform radius throughout and a solid surface free of indentations. The grip 200 can connect to an adapter 210, which can also define a hollow cylinder about an adapter axis AA, via a bridge portion 220. Similar to the previously described grip 4 and adapter 6, the grip 200 and adapter 210 can be offset from one another so the grip axis GA and adapter axis AA are non-coaxial and form an acute angle (not shown) therebetween. In such an embodiment, the adapter 210 can fit over a portion of a tattooing machine, such as the tattooing machine 30 shown in FIGS. 1-2, so the grip 200 can be linked to the needle 3 of the tattooing machine 30 and be non-coaxial with the needle axis NA defined by the needle 3, as previously described. The adapter 210 can also include one or more fastener openings 211 to receive fasteners, such as threaded screws, to tighten the adapter 210 to an attached tattooing machine. The bridge portion 220 connecting the grip 200 to the adapter 210 can include a first bridge end 221 connected to the grip 200 and a second bridge end 222 connected to the adapter 210, with the bridge portion 220 defining a curved shape between the first bridge end 221 and the second bridge end 222, as can be seen in FIG. 7. The curvature of the bridge portion 220 may be adjusted, as desired, to give the adapter 210, and therefore an attached tattooing machine, a desired offset from the grip 200. In other respects, the grip 200, adapter 210, and bridge portion 220 may be similar to the previously described grip 4, adapter 6, and bridge portion 5, respectively.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A tattooing system, comprising:
a needle defining a needle axis;
a needle driver connected to said needle and configured to reciprocally drive said needle along said needle axis; and
a grip linked to said needle and defining a grip axis which is non-coaxial with said needle axis.

2. The tattooing system according to claim 1, wherein said grip substantially defines a cylinder about said grip axis.

3. The tattooing system according to claim 2, wherein said cylinder includes a solid portion and a hollowed portion.

4. The tattooing system according to claim 3, wherein said solid portion includes at least one indentation formed therein.

5. The tattooing system according to claim 2, wherein said grip is completely hollowed.

6. The tattooing system according to claim 1, further comprising a gripping portion between said needle and said needle driver, said gripping portion defining a second grip axis which is coaxial with said needle axis.

7. The tattooing system according to claim 6, further comprising an adapter fit over said gripping portion and a bridge portion connecting said adapter to said grip.

8. The tattooing system according to claim 7, wherein said gripping portion defines a cylindrical shape.

9. The tattooing system according to claim 7, wherein said adapter defines a complementary shape to said gripping portion and includes at least one fastener configured to tighten said adapter to said gripping portion.

10. The tattooing system according to claim 7, wherein said bridge portion includes a first bridge end connected to said grip and a second bridge end connected to said adapter.

11. The tattooing system according to claim 10, wherein said bridge portion defines a curved shape between said first bridge end and said second bridge end.

12. The tattooing system according to claim 1, wherein said grip axis and said needle axis define an acute angle therebetween.

13. The tattooing system according to claim 12, wherein said acute angle is between 40 and 70 degrees.

14. The tattooing system according to claim 1, wherein said needle has a well formed therein.

15. A method of creating a marking in a skin layer of an animal using a tattooing system including a needle defining a needle axis and a needle driver connected to said needle, the method comprising:
loading said needle with a marking substance;
recriprocatingly driving said needle with said needle driver;
holding a grip linked to said needle and defining a grip axis which is non-coaxial with said needle axis; and
moving said grip such that said needle loaded with said marking substance goes into the skin of the animal to deposit said marking substance in the skin layer.

16. The method according to claim 15, wherein said grip is at least partially hollow.

17. The method according to claim 15, wherein said grip axis and said needle axis define an acute angle therebetween.

18. The method according to claim 17, wherein said acute angle is between 40 and 70 degrees.

19. The method according to claim 15, wherein said tattooing system further comprises a gripping portion between said needle and said needle driver, said gripping portion defining a second grip axis which is coaxial with said needle axis.

20. The method according to claim 19, wherein said tattooing system further comprises an adapter fit over said gripping portion and a bridge portion connecting said adapter to said grip.
